(19) United States
(12) Patent Application Publication
(10) Pub. No.: US 2013/0079892 A1
(43) Pub. Date: Mar. 28, 2013

(54) PROSTHETIC TUBE ADAPTER

(71) Applicant: Alexander IMHOF, BAYREUTH (DE)

(72) Inventor: Alexander IMHOF, BAYREUTH (DE)

(73) Assignee: MEDI GMBH & CO. KG, Bayreuth (DE)

(21) Appl. No.: 13/611,244

(22) Filed: Sep. 12, 2012

(30) Foreign Application Priority Data
Sep. 12, 2011 (DE) 10 2011 112 830.5

(51) Int. Cl. A61F 2/60

(52) U.S. Cl. CPC A61F 2/60
USPC 623/36

(57) ABSTRACT

Prosthesis tube adapter, comprising a tube and an adapter head, connected thereto, with a connecting sleeve which is fitted on the tube and in a connecting region is connected to this via an adhesive joint, wherein the connecting sleeve (8) and the tube (9) have complementarity inter-engaging, axially and radially abutting connection geometries in the connecting region in such a way that the prosthesis tube adapter (4) has a constant outside diameter right up to the region of the adapter head (7).

The invention relates to a prosthesis tube adapter, comprising a tube and an adapter head, connected thereto, with a connecting sleeve which is fitted on the tube acid in a connecting region is connected to this via an adhesive joint.

Such prostheses tube adapters usually serve for connecting an adapter counterpart—which is provided or; the body side, that is to say attached to the patient—to a prosthesis. One example is the connection between a lower leg shank, on which an adapter piece is provided, and a foot prosthesis. The wearer of the prosthesis, whose leg is amputated below the knee in this case, pulls the prosthesis shank over the leg stump. The adapter piece is located on the shank end. The prosthesis tube adapter is now detachably connected to this adapter piece by the adapter head. The foot prosthesis is then attached to the other end of the tube via suitable connecting pieces which are provided on the tube side and foot prosthesis side. Another exemplary application is the connection between a knee joint, on the lower end of which the adapter piece is located, and which is to be connected in turn so a foot prosthesis via the prosthesis tube adapter. These examples are not final, any other examples which require the use of a prosthesis tube adapter are naturally conceivable.

Known prosthesis tube adapters consist of a tube and an adapter head connected to this. The adapter head, for connecting purposes, has a connecting sleeve which is fixedly connected to the tube usually via an adhesive joint. To this end, the tube is usually inserted by its one end into the connecting sleeve, that is to say the connecting sleeve fits over the tube on the outside. The tube butts axially against the adapter head so that the insertion movement is limited. The fitting of the connecting sleeve over the tube is therefore chosen because as a result of this a large adhesion surface is created. In this case, however, it is disadvantageous that the outside diameter is increased in this region since here this is defined by the outside diameter, of the connecting sleeve. Many components which are to be attached to the tube and are matched to the outside diameter of the tube cannot consequently be pushed right up close to the adapter head since this obstructs the connecting sleeve. This, for example, is necessary where the tube—the length of which corresponds to the connecting distance to be bridged, to which it is exactly cut to length—is extremely short, for example in the case of the second exemplary application described, above in which the foot of the wearer of the prosthesis was amputated just above the foot joint, that is to say the adapter head is consequently located extremely close to the foot prosthesis which is to be attached. In this case, costly connecting techniques, or special solutions, are to be provided in order to be able to attach the foot prostheses via the prostheses tube adapter.

The possibility or inserting the connecting sleeve into the prosthesis tube so that the outside diameter of the tube is accessible up to the adapter head certainly exists. However, in this case a considerably reduced adhesion surface is created between the outer side of the connecting sleeve and inner side of the fate. This is disadvantageous since the adhesive joint especially plays an essential role with regard to the strength of the connection between the connecting sleeve and the tube, which is why a surface which is as large as possible is required in order to absorb the forces which occur during use and to keep the adhesive substance as free as possible from shear stresses and peeling stresses.

The invention is therefore based, on the problem of disclosing a prosthesis tube adapter which on the one hand makes it possible to have the outside diameter of the tube available right up to the adapter head, as well as to ensure a high-strength connection.

For solving this problem, it is provided according to the invention that the connecting sleeve and the tube have complementarity inter-engaging, axially and radially abutting connection geometries in the connecting region in such a way that the prosthesis tube adapter has a constant outside diameter right up to the region of the adapter head.

Whereas in the prior art cylindrical connection geometries are exclusively provided in the connecting region between connecting sleeve and tube, the invention provides a special connection geometry in the respective connecting region, wherein the two connection geometries are designed complementary to one another and inter-engage and abut both by an axial and a radial direction component. The outside diameter of the prosthesis tube adapter is the same in the entire region between the adapter head right up to the opposite tube end, that is to say is does not vary. That is to say that the entire length is available right up to a point adjacent to the adapter head in order to be able to attach to the tube corresponding components which are matched, to the tube outside diameter. Furthermore, according to the invention the connection between adapter head and tube is provided exclusively in the region between the connecting sleeve and the tube end without provision of an axial stop of the tube on the adapter head because the radial as well as the axial support between connecting sleeve and tube is provided by means of the corresponding, complementarily inter-engaging connection geometries which, furthermore, also have a sufficiently large connecting surface.

According to a first particularly advantageous embodiment of the invention, the connecting sleeve and the tube can have tapered, connecting surfaces in each case as connection geometries in the connecting region. According to this embodiment of the invention, the connecting sleeve and the tube are therefore interconnected via a scarf joint. Provision is made on the connecting sleeve as well as on the tube in the connecting region for corresponding tapered connecting surfaces which abut in the assembled position.

Since tapered surfaces are involved, an axial as well as a radial direction component are inevitably provided in the connecting region. This is advantageous for a particularly favorable force transfer from the adapter head to the tube, as a result of which weakening and loosening of the adhesive joint is obviated and the stability is increased compared with other connecting methods. Via the tapered or conical connecting surfaces a very large adhesion surface is made available, moreover, which is larger than in the case of a purely inserted connection. A further particular advantage of this type of connection, moreover, is the self-centering as a consequence of the inter-engaging conical shapes, as a result of which, a constant minimum adhesive gap is ensured between the tube and the connecting sleeve and the quality of the connection is improved as well as assembly being simplified.

In this case, the connecting sleeve can expediently overlap the tube on the outside. That is so say that the connecting sleeve widens in a conical or tapering manner on the inside from the adapter head towards the free end, whereas the tube becomes narrower in a conical or tapering manner, by its connecting end on the outside. Naturally, a reverse configuration would also be conceivable, that is to say that the connecting sleeve is inserted into the tube, therefore tapers on the outside, whereas the tube tapers on the inside.

For ensuring a maximum connecting surface, the sleeve-side, tapered connecting surface is expediently guided right up to the region of the adapter head. That is to say that the conical or tapered connecting surface extends from the free end of the connecting sleeve right up to the transition region to the adapter head. The tube-side, tapered connecting surface is naturally also correspondingly long.

In the case of the embodiment of the invention in which the connecting sleeve fits over the tube on the outside by the tapered connecting surface, the connecting sleeve has an outside diameter which is equal to the outside diameter of the tube so that any components can be pushed over the tube right info the region above the connecting sleeve. If the connecting sleeve is inserted into the tube by its outer tapered connecting surface, then the tube outside diameter consequently defines the given outside diameter. In both cases, however, the outstanding characteristics of the connecting technique according to the invention are retained.

An alternative to the described scarf joint provides that the connecting sleeve and the tube have in each case cylindrical connecting surfaces as connection geometries, which are serially delimited via a step in each case. In this alternative of the invention, the connection geometries again complementarily inter-engage and are axially and radially in abutment, but radially abutting cylindrical connecting surfaces as well as two serially abutting annular connecting surfaces in each case are provided in the region of the step abutments. In this case also, the connecting sleeve preferably fits over the tube on the outside, that is to say that the outside diameter of the connecting sleeve again corresponds to the outside diameter of the tube. The connecting sleeve is machined out in sections, that is to say is slightly enlarged from the inside diameter to an abutment step. Correspondingly complementarily to this, the tube is reduced in outside diameter in the connecting region on the outside, that is to say is machined down to an abutment step. If both are inserted one inside the other, then again a form-fitting, inter-engaging connection between the two is created, which in turn has high strength and at the same time ensures that a defined, constant outside diameter is provided right up to the adapter head.

Further advantages, features and individual characteristics of the invention are to be gathered from the exemplary embodiments described in the following text and also with reference to the drawings. In the drawings:

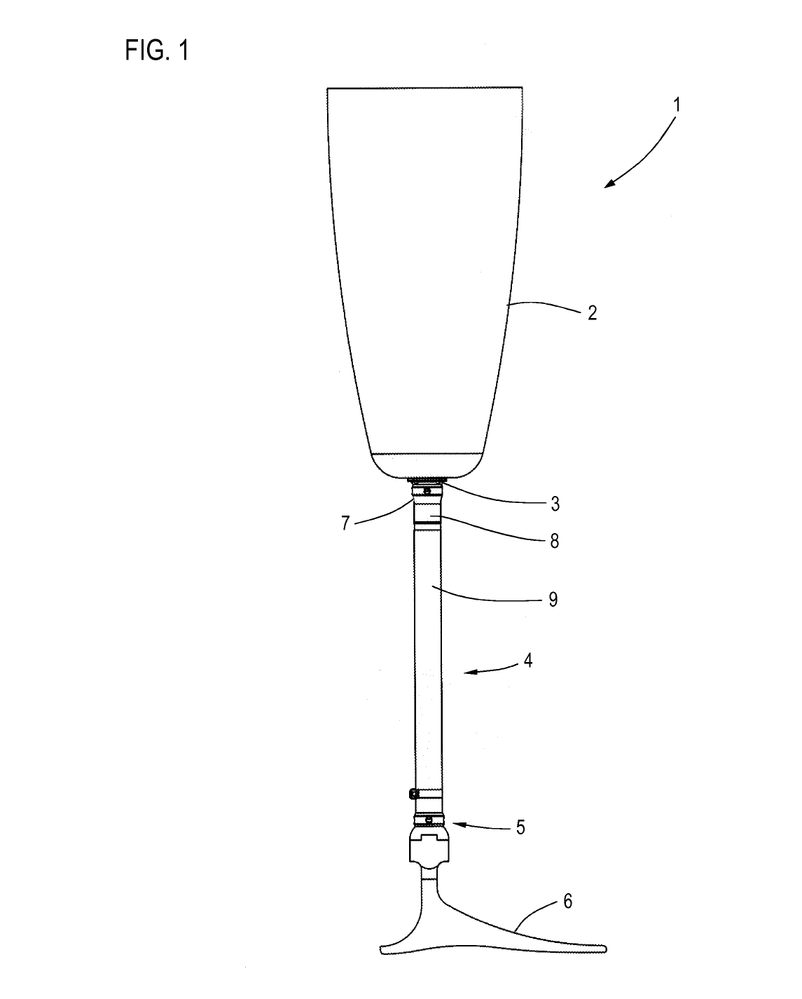

FIG. 1 shows an exemplary application for a prosthesis tube adapter according to the invention,

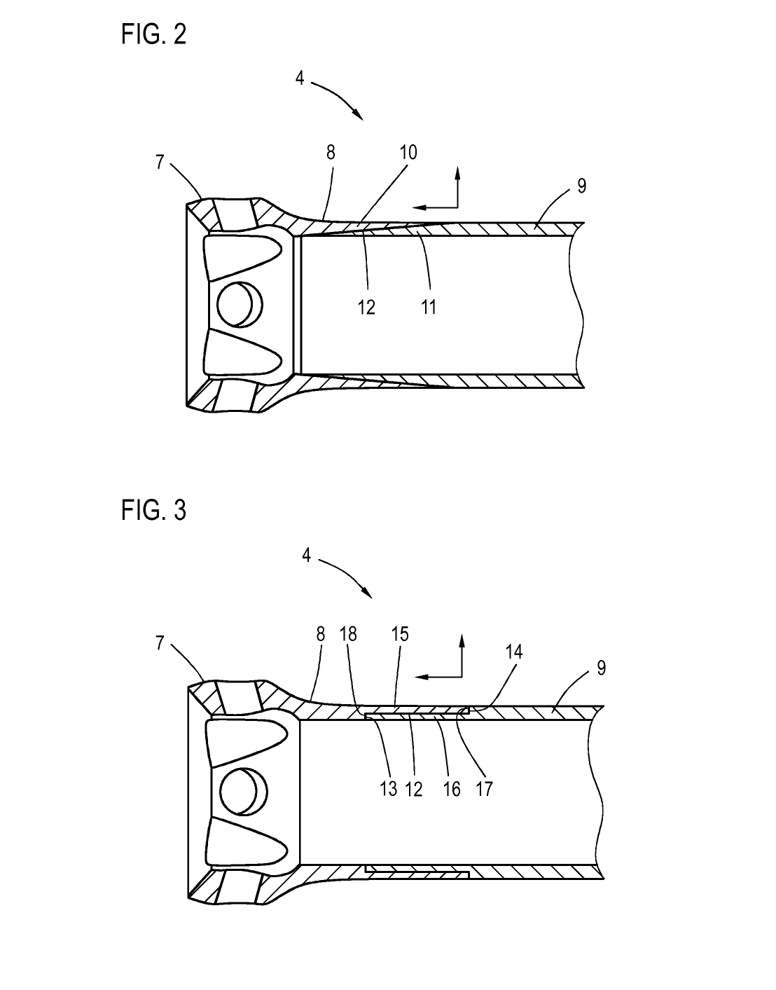

FIG. 2 shows a partial view of a prosthesis tube adapter according to the invention in the connecting region between adapter head or connecting sleeve and tube, in section,

FIG. 3 shows an exemplary application for a prosthesis tube adapter according to the invention according to a second embodiment.

FIG. 1 shows a schematic representation of a prosthesis 1, in this case a lower leg prosthesis, for example, comprising a shank 2, on which is arranged in a fixedly connected manner an adapter piece 3 to which is connected in turn the prosthesis tube adapter 4 according to the invention. Attached in turn to the lower end of the prosthesis tube adapter 4, via suitable connecting means 5, is a foot prosthesis 6.

The prostheses tube adapter 4 according to the invention consists of an adapter head 7, on which a connecting sleeve 8 is formed in one piece, this being expanded upon below, which connecting sleeve is connected to a tube 9, wherein the manner of connection is expanded upon below. The tube 9, for adapting the prosthesis 1, is correspondingly cut to length with its lower end which is to be connected to the foot prosthesis 6 later so that the prosthesis length can be accurately established.

The prosthesis tube adapter 4 clearly has a constant outside diameter, started from the lower end—to which the foot prosthesis 6 is attached via the connecting means 5—right up to the widening adaptor head 7 which is connected via suitable connecting means, such as screws or similar, to the adapter piece 3 of the shank 2. This makes it possible for any components, which are to be attached to the tube 9 and adjusted exactly to its outside diameter, to be able to be pushed right under the adapter head 7. This is necessary when the tube 9 is extremely short, for example, when the patient has lost the foot just above the foot joint and consequently the connection between adapter piece 3 and adapter head 7 is provided virtually directly above the foot prosthesis 6. Such a component, which is matched to the outside diameter, can be the connecting means 5, for example, which are fitted onto the tube 9 and fastened, to it.

A central element of the depicted prosthesis is the prosthesis tube adapter 4, as explained. This, as described, consists of the adapter head 7 on which is formed, in one piece, a connecting sleeve 8 which is cylindrical in outside diameter. In addition, the prosthesis tube adapter 4 also consists of the tube 9 which is fixedly connected to the connecting sleeve 8 via an adhesive joint.

This connection is shown according to a first embodiment in FIG. 2. In the case of this alternative of the invention, the connecting sleeve 8 and the tube 9 are adhesively bonded together via a so-cabled scarf joint. In this case, it entails a tapered surface fit. In the sectional view shown in FIG. 2, is evident that the connecting sleeve has a first connection geometry with a conical connecting surface 10. The inside diameter of the connecting sleeve 8 clearly increases continuously, beginning from the adapter head 7 towards the free sleeve end, so that a tapered or conical inner surface, which forms the connecting surface 10, is created.

The end of the tube 9 is formed geometrically complementarily to this. Also, the tube has a tapered connecting surface 11 which forms the connection geometry. In this case, however, the outside diameter of the tube 9 continuously decreases towards the end so that again a tapered or conical connecting surface 11 is created.

The respective lengths of the connecting surfaces 10, 11 are matched to one another so that in the assembled state, when they are interconnected via an adhesive layer 12 applied in between, they merge into each other essentially in a transition-free manner, which affects the outside diameter, or a stepless transition between the generated surface of the connecting sleeve 8 and the tube 9 is provided. In the case of the depicted embodiment of the invention, the connecting sleeve 8 fits over the tube 9 on the outside, the tube 9 extending virtually right up to the transition to the adapter head 7.

Via this tapered surface fit, a series of advantages is achieved.

So, for one thing, a larger adhesion surface is created as a result of the tapered, abutting surfaces, compared with a connection in which only cylindrical surfaces abut or are adhesively bonded together.

Furthermore, a particular advantage of the invention is that the outside diameter in the transition region from the tube 9 to the connecting sleeve 8 remains exactly the same. As a result of this, the total variance in the construction height of the prosthesis can be utilized. Furthermore, the scarf joint ensures a particularly favorable force transfer from the adapter head 7 to the tube 9. As a result of this, weakening and loosening of the adhesive joint is obviated and the stability compared with other connecting methods is improved. It is also advantageous in this case that the connecting surfaces 10, 11 are both axially and radially in abutment or support one another with an axial and a radial direction component, as shown by the arrow diagram in FIG. 2. This is again advantageous for force transfer or also for force distribution over the adhesion surface.

Finally, the conical shape of the scarf joint ensures automatic centering of the tube 9 in the connecting sleeve 8. As a result of this, a constant adhesive gap is ensured between tube 9 and connecting sleeve 8 and the quality of the connection is improved as well as assembly being made easier because as a result of the self-centering no special provisions are to be made for an accurate alignment.

Although FIG. 2 shows a configuration in which the tube 9 is inserted into the sleeve 8, a reverse configuration, in which the connecting sleeve 8 is inserted into the tube 9, is naturally also conceivable. The shape of the connection geometries, that is to say of the tapered connecting surfaces 10, 11, would then naturally be the other way round. The configuration shown in FIG. 2, however, is preferred.

FIG. 3 finally shows a further connection alternative according to true invention between the connecting sleeve 8 and the tube 9. In the depicted exemplary embodiment, the connecting sleeve 8 is again integrally constructed, that is to say in one piece with the adapter head 8. In this case also, the tube 9 engages by its end in the connecting sleeve 8.

The connecting sleeve 8 is enlarged by machining out from the inside diameter in the region of its connecting section. This region reaches from a step 13 up to the free end of the connecting sleeve 8.

In a corresponding, shape-complementary way, the tube 9 is reduced in outside diameter, again beginning at a step 14, right up to its free end, that is to say is therefore machined down. Therefore, in the case of this embodiment two hollow cylindrical connecting surfaces 15, 16 are created and are delimited in each case via a step 13 or 14. A cylindrical connecting surface 15 is consequently created on the connecting sleeve 8 in the region of its inner wall, while a cylindrical connecting surface 16 is provided on the tube 9 on its outer wall, being delimited in each case via steps 13 or 14.

It these connection geometries are now inserted one inside the other, then again a radial and also an axial abutment is brought about. The connecting surfaces 15, 16 radially abut and they define a minimum adhesive gap which in turn is filled with adhesive 12. The end-face edge 17 of the connecting sleeve 8 and the end-face edge 18 of the tube 9 butt axially against the corresponding steps 13, 14, from which results the axial support, as shown by means of the arrow diagram in FIG. 3. A favorable force transfer from the adapter head 7 into the tube 9 is again realised as a result of the both radial and axial support, provided on two end faces, between connecting sleeve 8 and tube 9.

At the same time, in this case also the outside diameter remains constant over the entire length right up to the adapter head because the outside diameter of the connecting sleeve 8 again corresponds to the outside diameter of the tube 9 so that a stepless transition is again created in the region of the connection.

In this cause also, there is naturally the possibility of configuring the connection in reverse in such a way that the connecting sleeve 8 is inserted into the tube 9, wherein in this case the connection geometries are then naturally again reversed.

1. Prosthesis tube adapter, comprising a tube and an adapter head, connected thereto, with a connecting sleeve which is fitted on the tube and in a connecting region is connected to this via an adhesive joint, characterized in that the connecting sleeve and the tube have complementarily inter-engaging, axially and radially abutting connection geometries in the connecting region in such a way that the prosthesis tube adapter has a constant outside diameter right up to the region of the adapter head.

2. Prosthesis tube adapter according to claim 1, wherein the connecting sleeve and the tube have tapered connecting surfaces in each case as connection geometries in the connecting region.

3. Prosthesis tube adapter according to claim 2, wherein the connecting sleeve fits over the tube on the outside.

4. Prosthesis tube adapter according to claim 2, wherein the sleeve-side, tapered connecting surface extends right up to the region of the adapter head.

5. Prosthesis tube adapter according to claim 1, wherein the connecting sleeve and the tube have in each case cylindrical connecting surfaces as the connection geometry, which are axially delimited via a step in each case.

6. Prosthesis tube adapter according to claim 5, wherein the connecting sleeve fits over the tube on the outside.

7. Prosthesis tube adapter according to claim 3, wherein the sleeve-side, tapered connecting surface extends right up to the region of the adapter head.
